(19) United States
(12) Patent Application Publication
(10) Pub. No.: US 2007/0225518 A1
(43) Pub. Date: Sep. 27, 2007

(54) 5-Aminolevulinic acid salts and their use

(76) Inventors: Zvi Malik, Kfar Haroe (IL); Abraham Nudelman, Rehovot (IL)

Correspondence Address: NATH & ASSOCIATES PLLC, 112 South West Street, Alexandria, VA, 22314, US

(21) Appl. No.: 11/386,092

(22) Filed: Mar. 22, 2006

(51) Int. Cl. C07F 9/22; C07C 229/22

(52) U.S. Cl. 562/8; 562/567; 562/84

(57) ABSTRACT

The invention provides salts of 5-Aminolevulinic acid (ALA) of formula (I): 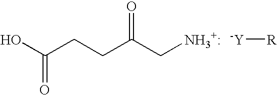
wherein RY is an organic acid moiety; Y is selected from the group consisting of a sulfonic acid residue, mono- or di-phosphoric acid residue, mono- or di-carboxylic acid residue and R is selected from the group consisting of saturated, unsaturated, straight or branched C₂-C₂₀ chains, aryl, aralkyl or naphthyl. In preferred embodiments, RY is selected from benzenesulfonic acid (besylate), 2-naphthalene sulfonic acid (napsyate), p-toluenesulfonic acid (tosylate), diethyl phosphate, dibenzyl phosphate, di-(2-ethylhexyl) phosphate, caproic or stearic acids. The invention also provides methods for preparing the ALA salts of the invention, pharmaceutical compositions containing the ALA salts of the invention, and use of the ALA salts of the invention in photodynamic therapy (PDT).

### FIELD OF THE INVENTION

This invention relates to 5-aminolevulinic acid (ALA) derivatives and their use.

### BACKGROUND OF THE INVENTION

5-Aminolevulinic acid (ALA), also known as 5-aminolaevulinic acid, δ-aminolevulinic acid, δ-aminolaevulinic acid, or 5-amino-4-oxopentanoic acid, is a naturally occurring amino acid that is an intermediate in the heme biosyngthesis pathway to the production of the endogenous photosensitizer, protoporphyrin IX (PpIX).

ALA is used in photodynamic therapy (PDT) as well as in cosmetic skin treatments. U.S. Pat. No. 5,955,490 describes the use of 5-ALA for treating acne. U.S. Pat. No. 6,710,066 describes the use of 5-ALA for treating non-malignant hyperproliferative skin lesions. In these applications, the ALA is administered topically to the skin and preferentially accumulates in target cells that are proliferating more rapidly than other cells in the target environment. The ALA is converted by endogenous cellular enzymes into protoporphyrin IX. The preferential accumulation of such naturally occurring porphyrins in rapidly growing cells permits the targeting of the cells. The target cells or tissue are then irradiated with light of the appropriate wavelength. Upon irradiation, the protoporphyrin IX is either made to fluoresce or to produce singlet oxygen, a highly cytotoxic compound. The target cells, containing sufficiently high concentrations of protoporphyrin IX, can thus be localized and distinguished from adjacent cells via fluorescence, or damaged or destroyed by the degradation products.

One of the difficulties in the use of ALA in PDT is its extreme instability. Aqueous solutions of ALA degrade quite rapidly, resulting in degradation products, primarily 2,5-pyrazine dipropionic acid. Formulating ALA in nonaqueous creams and gels does not prevent this degradation. Preparation of pharmacological equivalents of ALA such as functional derivatives of the carboxylic acid group, substitution of the amino group, blocking of the oxo group has not completely overcome this problem because the more stable the product, the greater effect there may be on the metabolism of the product inside the cells.

Because of its instability, ALA is most frequently used in PDT in the form of its hydrochloride salt (referred to herein as “ALA-HCl”). ALA-HCl and other known derivatives including salts are soluble in aqueous media but are insoluble in organic solvents including polyethylene glycol (PEG). Due to the insolubility of ALA-HCl in lipophilic media, when used for cosmetic purposes, it has to be provided as a dry powder and formulated in alcoholic medium immediately prior to topical administration. ALA-HCl has also been used in a pharmacologically equivalent form, such as an amide or ester.

U.S. Pat. No. 5,661,111 discloses acid addition salts of 5-ALA such as phosphate, nitrate, sulfate, acetate, propionate, butyrate, valerate, citrate, fumarate, maleate, malate and metal salts such as sodium, potassium and calcium salts used as an aqueous solution for improving plant salt tolerance.

U.S. Pat. No. 6,583,317 discloses a process of preparing an acid addition salt of ALA. A lower alkyl 5-bromolevulinate and hexamethylenetetramine are dissolved in a solvent selected from the group consisting of water, ethyl acetate, chloroform, acetone, ethanol, tetrahydrofuran and acetonitrile, to form a quaternary ammonium salt of the lower alkyl 5-bromolevulinate. The quaternary ammonium salt is then hydrolyzed with an inorganic acid to form an acid addition salt of ALA.

U.S. Pat. No. 6,034,267 discloses esters of ALA or pharmaceutically acceptable salts thereof for use in photochemotherapy or diagnosis. The esters are optionally substituted alkanols, i.e. alkyl esters or substituted alkyl esters.

### SUMMARY OF THE INVENTION

In its first aspect, the present invention provides novel salts of ALA. The ALA salts of the invention are soluble in aqueous as well as non-aqueous media, such as PEG, ethyl acetate, ethanol or propylalcohol. Solutions of the ALA salts of the invention have been found to be stable for prolonged periods both at 4° C. as well as at room temperature.

Thus, the present invention is directed to a compound of formula (I):

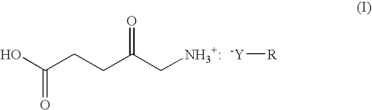

RY is an organic acid moiety wherein Y is selected from the group consisting of a sulfonic acid residue, mono- or di-phosphoric acid residue, mono- or di-carboxylic acid residue and R is selected from the group consisting of saturated, unsaturated, straight or branched C₂-C₂₀ chains, aryl, aralkyl or naphthyl. Preferably the organic acid is selected from the group consisting of saturated and unsaturated, straight or branched C₂-C₂₀ chains sulfonic acid; C₆ to C₂₀ aliphatic mono- and di-carboxylic acids; mono- and di-C₂-C₂₀ straight or branched aliphatic phosphoric acid esters and their salts.

Most preferably the organic acid is selected from benzenesulfonic acid (besylate), 2-naphthalene sulfonic acid (napsylate), p-toluenesulfonic acid (tosylate), diethyl phosphate, dibenzyl phosphate, di-(2-ethylhexyl) phosphate, caproic or stearic acids.

The invention is yet further directed to a process for the preparation of compounds of formula (I). In a first embodiment, the process involves reacting in an organic solvent under N₂ at room temperature, N-Boc-5-aminolevulininc acid residue with an organic acid of formula RY as defined above, to yield the desired compound of formula (I):

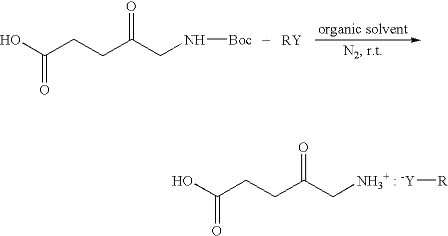

In a second embodiment the process involves hydrogenation of 5-nitrolevulininc acid in the presence of an organic acid of formula RY as defined above to yield the desired compound of formula (I):

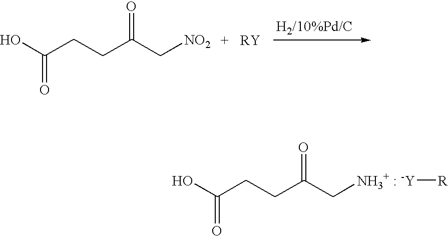

Preferably, the organic acid is selected from sulfonic acids. In particular, the benzenesulfonic acid (besylate) and the 2-naphthalene sulfonic acid (napsylate) salts of ALA have been synthesized and found to be soluble and stable in aqueous as well as non-aqueous media such as PEG.

As shown below, the ALA salts of the invention are taken up by cells and converted into protoporphyrin IX. Irradiation of the cells following uptake with irradiation at a wave length in the range of 360-410 nm leads to a decrease in cell viability.

In other of its aspects, the invention provides solutions of the ALA salts of the invention. The solutions may be aqueous solutions, or non-aqueous solutions, such as PEG solutions.

In another of its aspects, the invention provides pharmaceutical compositions comprising an ALA salt of the invention and a pharmaceutically acceptable carrier. The pharmaceutical composition may be in a form suitable for topical application to skin, such as a cream or gel.

In another of its aspects, the invention provides a method of treating cells. In accordance with this aspect of the invention, ALA salts of the invention are administered to the cells in a manner that allows the cells to take up the ALA salts. The cells are then illuminated with illumination in a range of wavelengths absorbed by protoporphyrins formed inside the cells. In one embodiment, the intensity of the illumination is selected to cause the protoporphyrins to fluorescence. In another embodiment the intensity of the illumination is selected to cause protoporphyrins formed inside the cells to be degraded. The method of the invention may be used to achieve destruction of cells by the formation of degradation products of the protoporphyrins formed in the cells. The cells may be for example, skin cells, or subcutaneous cells. In this case, a pharmaceutically composition containing one or more compounds of the invention are applied to the skin surface. The pharmaceutical compositions may be in the form of a cream or lotion. The method may be used for dermatologically or cosmetically treating cells. The method of the invention may be used in photodynamic therapy (PDT).

Thus, in one of its aspects, the invention provides a compound of formula (I):

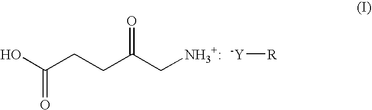

wherein RY is an organic acid moiety; Y is selected from the group consisting of a sulfonic acid residue, mono- or di-phosphoric acid residue, mono- or di-carboxylic acid residue and R is selected from the group consisting of saturated, unsaturated, straight or branched C₂-C₂₀ chains, aryl, aralkyl or naphthyl. A compound according to claim 1 wherein RY is selected from the group consisting of saturated and unsaturated, straight or branched C₂-C₂₀ chains sulfonic acid; C₆ to C₂₀ aliphatic mono- and di-carboxylic acids; mono- and di-C₂-C₂₀ straight or branched aliphatic phosphoric acid esters and their salts.

In another of its aspects, the invention provides a process for the preparation of a compound of formula (I) comprising:

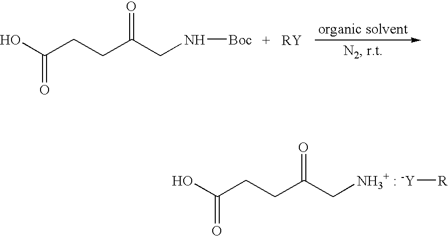

In still another of its aspects, the invention provides a process for the preparation of a compound of formula (I) comprising:

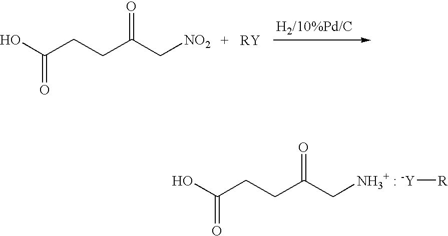

The invention also provides a solution comprising a compound of the invention.

The invention still further provides a pharmaceutical composition comprising a compound of the invention and a pharmaceutically acceptable carrier.

In yet another of its aspects, the invention provides a method of treating cells comprising administering a compound of the invention to the cells under conditions allowing the cells to take up the compound.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows PpIX formation in by HPLC following exposure to (a) ALA-HCl, (b) AN-301, and (c) AN-302;

FIG. 2 shows PpIX formation by spectroscopy following exposure to (a) ALA-HCl, (b) AN-301, and (c) AN-302;

FIG. 3 shows porphyrin formation by flow cytometry in Friend erythroleukemia cells (FLC) following uptake of AN-301 (FIG. 3A) and AN-302 (FIG. 3B) from stock solutions of the ALA salt made up in (a) H₂O, (b) PEG, and (c) buffer pH7, in comparison with controls (d) not presented with an ALA salt;

FIG. 4 shows porphyrin formation by flow cytometry in Friend erythroleukemia cells (FLC) following uptake of AN-301 (FIG. 3A) and AN-302 (FIG. 3B) from stock solutions that had been stored for 7 days at 4° C. (a), 37° C. (b), or after no storage (c) in comparison with controls (d) not presented with an ALA salt; and

FIG. 5 shows survival of B16 melanoma cells following presentation of an ALA salt (ALA-C1, AN-301 or AN-302) and irradiation.

### DETAILED DESCRIPTION OF THE INVENTION

As mentioned above, the present invention concerns novel compounds of formula (I) which are organic acid salts of ALA. These salts are soluble in organic medium and are stable for prolonged periods both at room temperature and as low as 4° C. The solubility and the prolong stability of the compounds of the present invention facilitate the use of such compounds for therapeutic purposes.

### Preparation of Compounds of the Invention

### EXAMPLE 1

### 1: 5-Amino4-oxopentanoic acid benzenesulfonate (ALA-besylate, also referred to herein as “AN-301”)

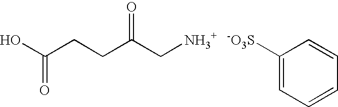

A solution of N-Boc-5-aminolevulinic acid (1 eq) and benzenesulfonic acid (1 eq) in dry CH₂Cl₂ under N₂ was stirred for 4 h at rt. Water (20 mL) was then added, the aqueous layer was washed with CH₂Cl₂ and evaporated to give 5-Amino-4-oxopentanoic acid benzenesulfonate as a white solid (80% yield), mp 169-171° C. 1H-NMR (300 MHz, MeOD) ppm δ 2.63 (t, J=6.3 Hz, 2H, CH₂CO₂), 2.77 (t, J=6.1 Hz, 2H, CH₂CO), 4.01 (s, 2H, CH₂NH₂), 7.43 (m, 3H, 2Hₘ, Hₚ), 7.81-7.85 (m, 2H, 2Hₒ). ¹³C-NMR (300 MHz, MeOD) ppm [symbol] 28.37 (CH₂CO₂), 35.33 (CH₂CO), 48.1 (CH₂NH₂), 126.8 (Cₒ), 129.4 (Cₘ), 131.4 (Cₚ), 146.0 (Cᵢ), 175.9 (CO₂), 203.1 (CO). MS (ES+): m/z 114 (MH⁺—H₂O, 100). MS (ES−): m/z 157 (M⁻, 100).

### EXAMPLE 2

### 5-Amino-4-oxopentanoic acid 2-naphtylsulfonate (ALA-napsylate, also referred to herein as “AN-302”)

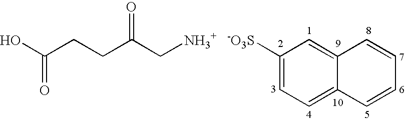

A solution of N-Boc-5-aminolevulinic acid (1 eq) and 2-naphthylsulfonic acid (1 eq) in dry CH₂Cl₂ under N₂ was stirred for 4 h in rt. Water (20 mL) was then added, and the aqueous layer was washed with CH₂Cl₂ and evaporated to give 5-amino-4-oxopentanoic acid 2-napthylsulfonate as a white solid (68% yield), mp 172-175° C. ¹H-NMR (300 MHz, MeOD) ppm δ2.61 (t, J=6.3 Hz, 2H, CH₂CO₂), 2.74 (t, J=6.1 Hz, 2H, CH₂CO), 4.01 (s, 2H, CH₂NH₂), 7.56 (m, 2H, H6, H₇), 7.9 (m, 4H, H₃, H₄, H₅, H₈), 8.63 (m, 1H, H₁). ¹³C-NMR (300 MHz, MeOD)ppm [symbol] 28.3 (CH₂CO₂),35.3 (CH₂CO), 48.1 (CH₂NH₂), 124.0 (C₃), 126.4 (C₄), 127.9 (C₁), 128.5 (C₇), 128.8 (C₆), 129.3 (C₈), 128.8 (C₅), 132.2 (C₂), 133.8 (C₉), 135.3 (C₁₀), 175.8 (CO₂), 203.1 (CO). MS (ES+): m/z 114 (MH⁺−H₂O, 100). MS (ES−): m/z 207 (M⁻, 100).

### BIOLOGICAL EXPERIMENTS

### Materials and Methods

Cell culture. Friend erythroleukemia cells (FLC) and B16 melanoma cells were grown in a DMEM medium and RPMI 1640, respectively, (Biological Industries, Beit-Haemek, Israel), supplemented with 10% fetal calf serum and antibiotics, on tissue culture plates (Corning, Cambridge, Mass., USA) and incubated at 37° C. in a humidified atmosphere with 8% CO₂. The cells were re-cultured twice a week.

Extraction of porphyrins from cells. FLC were incubated with 0.1 mg/ml ALA-HCl, AN 301, or AN 302 for 4 h. The cells were then washed and suspended in 750 μl glacial acetic acid. The cells were then sonicated on ice, at an amplitude of 1-2, three times for 45 sec, with 15 sec between successive sonications. Following the sonication, the lysate was transferred to glass test tubes containing 2.25 ml ethyl acetate, vigorously vortexed and centrifuged for 15 min at 3000 rpm. 2.6 ml of the supernatant were then transferred to glass test tubes containing 500 μl/1000 μl 1 M HCl and incubated overnight at 4° C. in the dark. Two phases formed, a lower inorganic phase and an upper organic phase containing the porphyrins. 300-700 μl from the organic phase was carefully removed with a Pasteur pipet. The porphyrins were then examined by HPLC and by spectroscopy.

High pressure liquid chromatograph [HPLC] analysis of porphyrins. Extracted porphyrins were purified by HPLC on an Apex Presil C18 8μ column (Jones, Chromatography, Lakewood, Colo.) using a 0-50% linear gradient aetonitrile (+0.1% trifluoroacetic acid) over 40-min elution at 47% acetonitrile.

Fluorometry of porphyrins. Fluorescence spectroscopy of the extrated porphyrins was examined using a SLM-Aminco spectrometer. Emission spectra were recorded following excitation at 410 nm.

Flow cytometry for cellular PpIX. FLC were grown on tissue culture plates and were incubated with 0.1 mg/ml ALA-HCl, AN-301, or AN-302 for 4 h. After the incubation, the cells were washed with Ca⁺²—Mg+² free PBS and scraped off with a rubber policeman. After a 10 min centrifugation at 1100 RPM, the supernatant was decanted and the pellet was resuspended in 0.5 ml Ca⁺²—Mg⁺² free PBS. The cell suspension was filtered and measured using a Fluorescence Activated Cell Sorter (Becton Dickinson FACS Calibur, Mountain View, Calif., USA). 10,000 cells were measured in each sample using an excitation wavelength of 488 nm, and emission >600 nm, respectively.

ALA treatment and photosensitization. B15 melanoma cells were cultured for 24 h with serum and the medium was then replaced in the dark by serum free medium, with 0.6 mM ALA (Sigma), AN-301, or AN-302 for 4 hr. At the end of the incubation period, the cells were irradiated using a Vilber Lourmat lamp, VL-206BL, delivering a power density of 22.5 mW at 360-410 nm. Light intensity was measured with a Nova photometer (Ophir Optronics, Jerusalem).

MTT assay. The effect of PDT on cell viability was measured after 24 hr. by a modified MTT assay which is based on the ability of live cells to cleave the tetrazolium ring to a molecule that absorbs at 590 nm in active mitochondria (Mosmann, 1983). 2×10³ cells were grown in 96-well plates. The growth medium was replaced and 20 μl MTT (3-(4,5-dimethylthiazol-2-yl)-2,5-diphenyl tetrazolium bromide), 5 mg/ml PBS (Sigma, Chemical Co, St. Louis, Mo., USA), were added to each well. The cells were further incubated at 37° C. for 2 h. For lysis of the cells, N,N-dimethyformamide was added to the media for 5 hours and absorbance was then measured at 590 nm.

Results

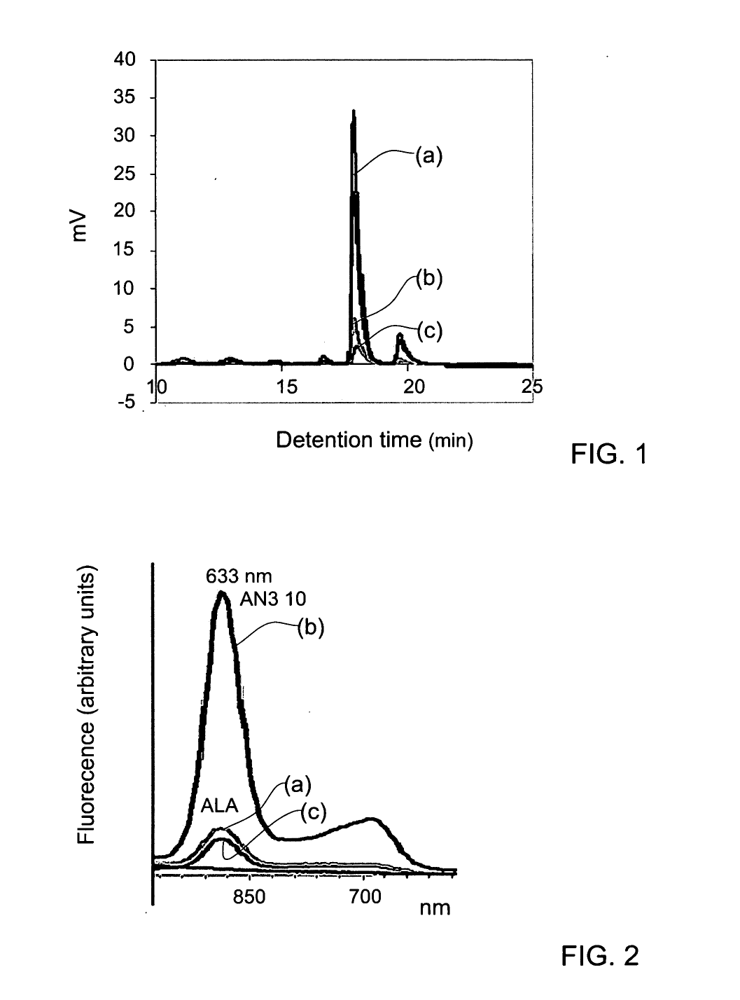

FLC cells were incubated in the presence of ALA-HCl, AN-301 or (c) AN-302 and analyzed for PpIX formation by HPLC and spectroscopy, as described above. FIG. 1 shows PpIX formation in the cells as determined by HPLC. The retention time on the column, which was similar in all three treatments, is typical of PpIX showing that the same porphyrin (PpIX) was generated by exposure to (a) ALA-HCl, (b) AN-301, and (c) AN-302. FIG. 2 shows PpIX formation in the cells by spectroscopy. A Characteristic major peak at 633 was observed in all three cases, confirming that the same porphyrin product was generated by exposure to (a) ALA-HCl, (b) AN-301 and (c) AN-302.

Stock Solutions

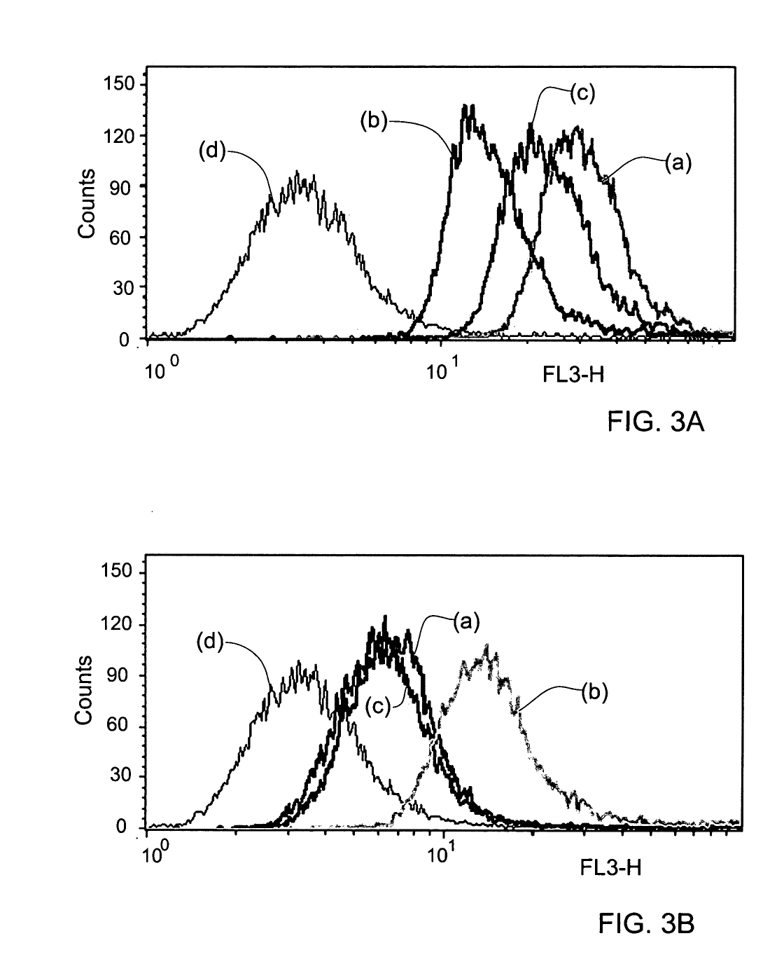

FIG. 3 shows biosynthesis of protoporphyrin PpIX following uptake by FLC of AN-301 (FIG. 3A) and AN-302 (FIG. 3B). Stock solutions of the ALA salt were made up in (a) H₂O, (b) PEG, and (c) buffer pH7. Both AN-301 and AN-302 were found to be soluble in each of these three solvents. The ALA salt was then presented to the cells, and biosynthesis of PpIX was determined by flow cytometry. In controls (d) no ALA salt was presented to the cells. Typical results are shown in FIG. 3 in which the horizontal axis is florescence intensity (in arbitrary units), and the vertical axis is the number of cells having a given intensity level. The results show that treatment of the cells with AN-301 or AN-302 leads to the formation of PpIX inside the cells.

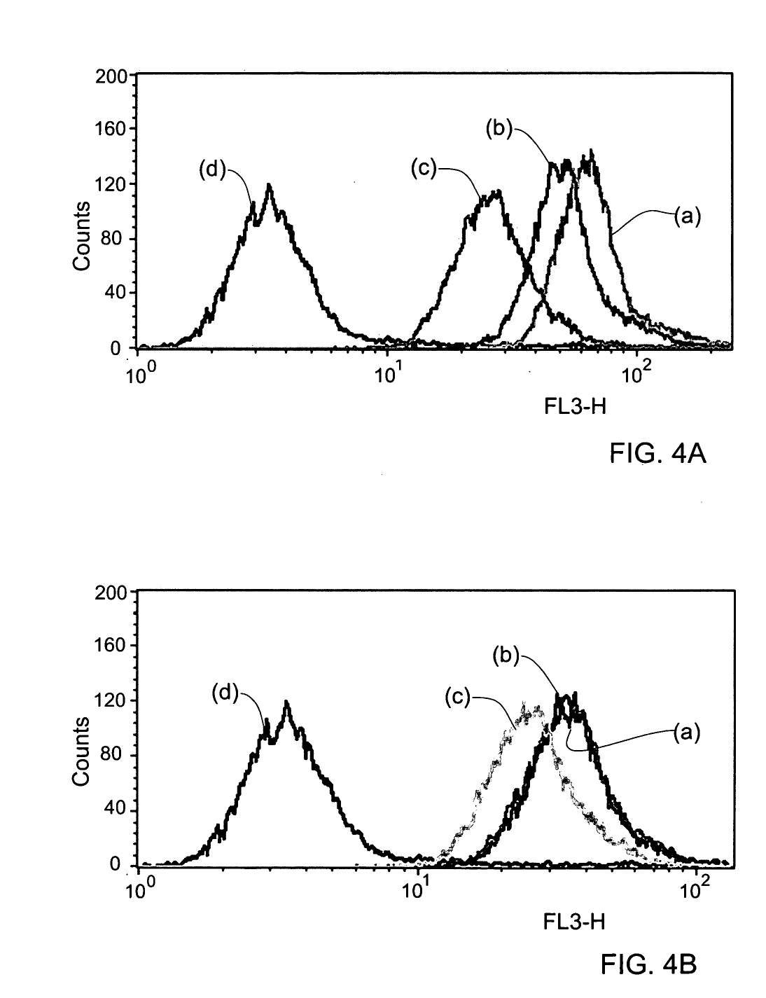

FIG. 4 shows the effect of storage on the stability of AN-301 and AN-302. Solutions of AN-301 (A), AN-302 (B) were prepared at a concentration of 0.1 mg/ml, and presented to FLC cells after being stored for 7 days at 4° C. (a), 37° C. (b), or after no storage (c). In controls (d), no ALA salt was presented to the cells. The cells were then examined for the presence of PpIX by flow cytometry at 360 nm. Typical results are shown in FIG. 4. The results show that the AN-301 and AN-302 solutions were stable for 7 days at 4° C., as well as at 37° C.

Table 1 shows the stability of ALA-HCl, AN-301 and AN-302 during storage, as demonstrated in FIG. 4. The table shows porphyrin formation in arbitrary units in FLC determined by flow cytometry following incubation of the cells in the presence of an ALA salt from a stock solution previously stored as indicated in the table. ALA-HCl is completely degraded after 7 days of storage at either 37° C. or at 4° C., as evidence by the absence of porphyrin formation in the cells. In contrast to this, the compounds of the invention, AN-301 and AN-302, retained significant activity after 7 days of storage at both 4° C. and 37° C., as evidence by significant porphyrin formation in the cells.

TABLE 1

| Storage time | | | |
|---|---|---|---|
| and temperature | ALA-HCl | AN-301 | AN-302 |
| 0 | 60 | 57 | 49 |
| 7 days, 37° C. | 3.5 | 39 | 29 |
| 7 days, 4° C. | 3.5 | 39 | 23 |

Table 2 summarizes the properties of AN-301 and AN-302. Both compounds were taken up by cells from water similar to the take up of ALA-HCl from water by the cells. Neither compound was found to be stable in water for seven days, but they were both found to be stable in PEG for 7 days. Both compounds generated porphyrin synthesis in the cells, even after seven days of storage. Both compounds had a solubility in water similar to that of ALA-HCl. However, unlike ALA-HCl, AN-301 and AN-302 were soluble in PEG.

TABLE 2

| | (b) | (c) | (d) |
|---|---|---|---|
| (a) | ALA-HCl | AN-301 | AN-302 |
| Water solubility | + | + | + |
| Lipid solubility | − | + | + |
| PEG | | | |
| Uptake by cells | + | + Stock | + Stock |
| from water | | solution in | solution in |
| | | PEG | PEG |
| Stability in H₂O | − | − | − |
| (7days) | | | |
| Stability in PEG | Not | + | + |
| (7days) | soluble in | | |
| | PEG | | |
| Porphyrin | + | + | + |
| synthesis | | | |
| Porphyrin | − | + | + |
| synthesis from | | | |
| PEG (7 days) | | | |

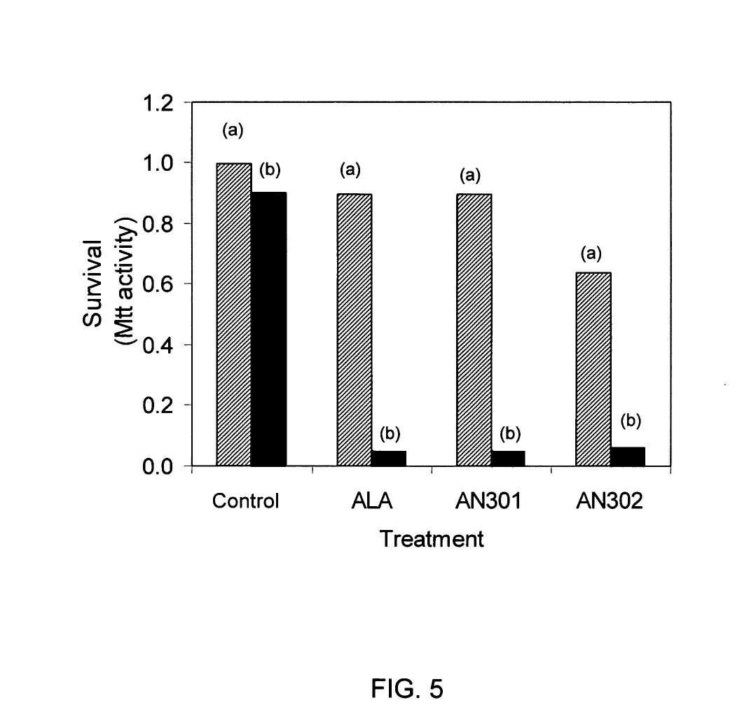

FIG. 5 shows the results of the photodynamic therapy on B16 melanoma cells. Cell cultures were presented with ALA-HCl, AN-301 or AN-302. In control cultures, the ALA salt was omitted. Comparison of MTT activity following illumination of cultures (bars (b)) with corresponding cultures that were not illuminated (bars (a)) shows that AN-301 and AN-302 were as effective as ALA-HCl in causing cell death. In the control cultures, the illumination only had a slight effect on cell survival.

1. A compound of formula (1): 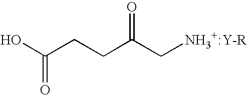
wherein RY is an organic acid moiety; Y is selected from the group consisting of a sulfonic acid residue, mono- or di-phosphoric acid residue, mono- or di-carboxylic acid residue and R is selected from the group consisting of saturated, unsaturated, straight or branched C₂-C₂₀ chains, aryl, aralkyl or naphthyl. A compound according to claim 1 wherein RY is selected from the group consisting of saturated and unsaturated, straight or branched C₂-C₂₀ chains sulfonic acid; C₆ to C₂₀ aliphatic mono- and di-carboxylic acids; mono- and di-C₂-C₂₀ straight or branched aliphatic phosphoric acid esters and their salts.

2. A compound according to claim 1 wherein RY is selected from benzenesulfonic acid (besylate), 2-naphthalene sulfonic acid (napsylate), p-toluenesulfonic acid (tosylate), diethyl phosphate, dibenzyl phosphate, di-(2-ethylhexyl) phosphate, caproic or stearic acids.

3. A compound according to claim 1 wherein RY is benzenesulfonic acid, p-toluenesulfonic acid, naphthalenesulfonic acid.

4. A process for the preparation of a compound of formula (I) comprising:

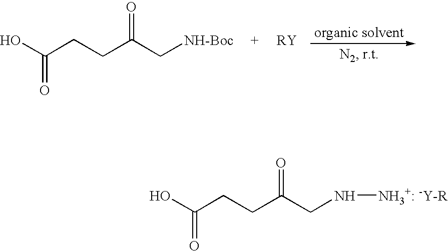

5. A process for the preparation of a compound of formula (I) comprising:

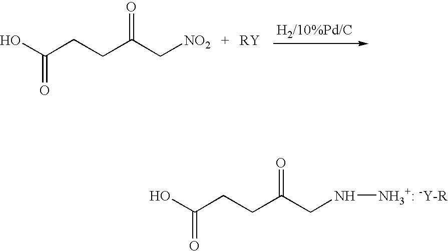

6. A solution comprising a compound according to any one of claims 1 to 4 and a solvent.

7. The solution according to claim 6 wherein the solvent is water.

8. The solution according to claim 6 wherein the solvent includes an organic compound.

9. The solution according to claim 8 wherein the solvent includes a compound selected from the group comprising polyethylene glycol (PEG), ethyl acetate, ethanol and propylalcohol.

10. A pharmaceutical composition comprising a compound according to claim 1 and a pharmaceutically acceptable carrier.

11. The pharmaceutical composition according to claim 10 in a form suitable for topical application to skin.

12. The pharmaceutical composition according to claim 11 in the form of a cream or gel.

13. A method of treating cells comprising administering a compound according to claim 1 to the cells under conditions allowing the cells to take up the compound.

14. The method according to claim 13 further comprising illuminating the cells with illumination in a range of wavelengths absorbed by protoporphyrins.

15. The method according to claim 14 wherein the illumination has an intensity selected to cause the protoporphyrins to fluorescence.

16. The method according to claim 14 wherein the illumination has an intensity selected to cause the protoporphyrins to be degraded.

17. The method according to claim 14 further comprising destruction of cells by degradation products of the protoporphyrins formed in the cells.

18. The method according to claim 14 wherein the cells are skin cells or subcutaneous cells.

19. The pharmaceutical composition according to claim 10 for use in localizing cells.

20. The pharmaceutical composition according to claim 11 for use in photodynamic therapy (PDT).

21. The method according to claim 13 wherein the compound is applied to a skin surface.

22. The method according to claim 21 wherein the compound is in a form selected from the group comprising a cream or lotion.

23. The method according to claim 13 for dermatologically or cosmetically treating skin.
